(19) United States
(12) Patent Application Publication
(10) Pub. No.: US 2024/0294935 A1
(43) Pub. Date: Sep. 5, 2024

(54) RESISTANCE GENE AND LETTUCE PLANT RESISTANT TO DOWNY MILDEW

(71) Applicant: Enza Zaden Beheer B.V., Enkhuizen (NL)

(72) Inventors: Bas Ter Riet, Medemblik (NL); Geert Johannes De Boer, IJmuiden (NL); Ilja Roobeek, Dirkshorn (NL); Mathieu André Pel, Enkhuizen (NL)

(21) Appl. No.: 18/427,141

(22) Filed: Jan. 30, 2024

Related U.S. Application Data
(62) Division of application No. 17/268,540, filed on Feb. 15, 2021, now Pat. No. 11,920,144.

(51) Int. Cl. C12N 15/82; A01H 6/14; C07K 14/415

(52) U.S. Cl. CPC C12N 15/8282; A01H 6/1472; C07K 14/415

(57) ABSTRACT

The present invention relates to a lettuce plant that is resistant to downy mildew, more specifically to a lettuce plant that comprises a mutated gene that confers broad spectrum resistance to oomycetes in lettuce. Furthermore the present invention relates a resistance gene and a method for obtaining a lettuce plant that is resistant to downy mildew, wherein the method comprises the step of mutating a gene.

### CROSS-REFERENCE TO RELATED APPLICATIONS

This application is a Divisional of U.S. patent application Ser. No. 17/268,540, filed Feb. 15, 2021, which is a U.S. National Stage Application of International Application No. PCT/EP2018/072249, filed Aug. 16, 2018, each of which is incorporated herein by reference in its entirety.

### SEQUENCE LISTING

A Sequence Listing in the form of an XML file (entitled “EAB-29201_SL.xml”, created on Apr. 30, 2024, and having a size of 18,522 bytes) is hereby incorporated by reference in its entirety.

### DESCRIPTION

The present invention relates to a lettuce plant that is resistant to downy mildew, more specifically to a lettuce plant that comprises a mutated gene that confers broad spectrum resistance to oomycetes in lettuce. Furthermore the present invention relates a resistance gene and a method for obtaining a lettuce plant that is resistant to downy mildew, wherein the method comprises the step of mutating a gene.

Downy mildew refers to several types of oomycete microbes that are parasites of plants. Downy mildew can originate from various species, but mainly of Peronospora, Plasmopara and Bremia. Downy mildew is a problem in many food crops, in for example in lettuce caused by Bremia lactucae, affecting the production of this crop worldwide. Plants that are being affected include food crops such as brassicas (e.g. cabbage), potatoes, grape, spinach, lettuce, onion, tomato, cucumber plants. Downy mildew infection show symptoms of discoloured areas on upper leaf surfaces in combination with white, grey or purple mould located on the other side of the leaf surface below. Disease is spread from plant to plant by airborne spores.

Lettuce, mostly known as Lactuca sativa, but also including Lactuca species such as L. serriola, L. saligna or L. virosa, is a very important crop worldwide. Some of the most popular varieties available are Iceberg, Romaine, Butterhead, Batavia and Oakleaf. There are many plant pathogens that affect L. sativa, and some of the diseases caused by these pathogens are downy mildew, sclerotinia rot, powdery mildew, fusarium wilt of which the most important disease is lettuce downy mildew, which is caused by the B. lactucae, an oomycete pathogen that belong to Peronosporaceae.

For some vegetable crops, such as lettuce, cultivars with resistance to downy mildew are available. However, the pathogen under pressure will mutate to break down the disease resistance and new disease resistance in crops is needed to control infection. Especially in lettuce the occurrence of resistant downy mildew is particularly complex as there are many different races, and new resistant downy mildew species emerging all the time.

In lettuce, infection of B. lactucae result in yellow to pale green lesions that eventually become necrotic due to secondary pathogens leading to major crop losses. Fungicides can be used to control B. lactucae, but eventually B. lactucae becomes immune to these chemicals, because over time the pathogen also acquires resistance to fungicides. Furthermore, there are multiple lettuce varieties available that are resistant to B. lactucae but resistance is quickly overcome because new Bremia races develop rapidly. Therefore, it is of the utmost importance to find other methods to control B. lactucae infection. Most preferably is to identify a resistance gene that gives broad resistance against B. lactucae and to provide for lettuce plants that are resistant to downy mildew. Therefore, identification of resistance genes is a promising alternative.

### SUMMARY

Considering the above, there is a need in the art for to provide plants that are resistant to downy mildew and wherein plants have a broad spectrum resistance against this pathogen. Furthermore, it is an object of present invention to provide a method to obtain such downy mildew resistant plants.

It is an object of the present invention, amongst other objects, to address the above need in the art. The object of present invention, amongst other objects, is met by the present invention as outlined in the appended claims.

### BRIEF DESCRIPTION OF THE DRAWINGS

The present invention will be further detailed in the following examples and figures wherein:

FIG. 1: shows leaves of L. sativa plants that are resistant (left) or susceptible (right) to Bremia lactucae. All lettuce plants comprised the MACPFR1 resistance gene. Subsequently this MACPF1R gene has been silenced in these plants using VIGS gene silencing and infected with Bremia lactucae. As expected with transient gene silencing, VIGS gene silencing does not result in fully 100% silencing of the MACPF1R gene in all plants. The leaves on the right originate from plants, wherein the resistance gene has been silenced by VIGS silencing, therefore the MACPF1R gene expression was reduced. The leaves on the left originate from plants wherein VIGS silencing was less successful in reducing the MACPF1R gene expression, therefore there is still a significant amount of MACPF1R gene expression in these plants. Leaves of the plants (right) wherein the resistance gene was silenced showed to be susceptible to downy mildew. The MACPF1R expression levels in the leaves of both groups (plants that showed to be resistant or susceptible to downy mildew) were collected and RNA was isolated to determine the expression levels of the resistance gene by qPCR (see FIG. 2).

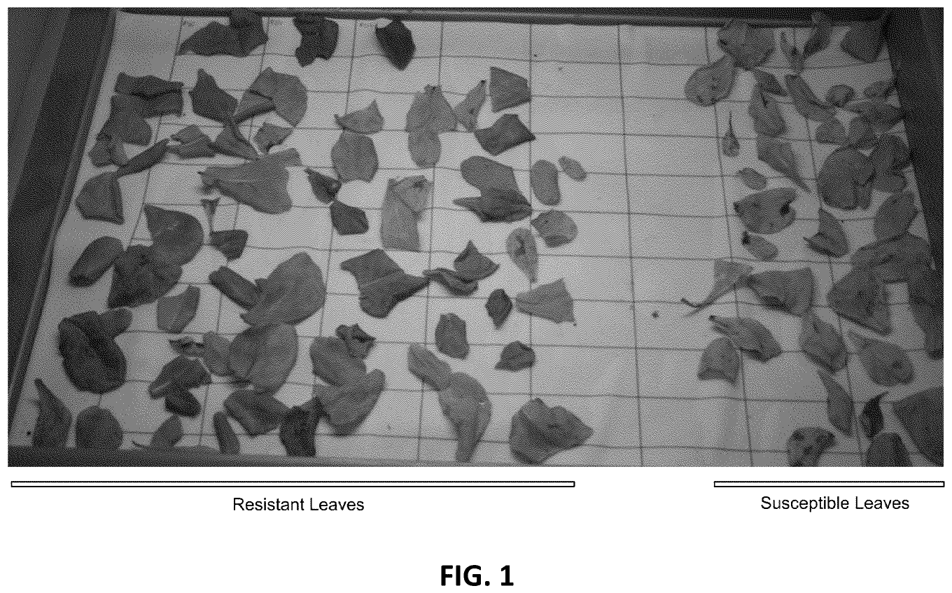

FIG. 2: shows MACPF1R expression levels in MACPF1R VIGS silenced lettuce lines infected with Bremia (B130). The transcription levels, determined by qPCR, of MACPF1R resistance gene in relation to the transcription levels of a house keeping gene (TUA-3). The transcription levels of Bremia lactuca was determined by the transcripts of a Bremia house keeping gene (Actin) in relation to the lettuce house keeping gene TUA-3 of Bremia lactucae were determined in leave samples of L. sativa plants of the experiment of FIG. 1. Leaves of the plants that were resistant to Bremia lactucae showed to have a high MACPF1R gene expression and low transcriptional levels of the Bremia lactucae house keeping gene. Leaves of the plant that were susceptible to Bremia lactucae, showed low MACPF1 gene expression and high transcriptional levels of the Bremia lactucae house keeping gene, indicating the susceptibility corresponds with low MACPF1R gene expression.

FIG. 3: shows an overview of the disease test performed with the most recent isolates of Bremia B116 to 33 on L. sativa lines Cobham Green, Green Towers, Vanity and SE01. SE01 is a lettuce plant (L. sativa) of present invention comprising the MACPF1R resistance gene. The plant of present invention shows to be fully resistant to all downy mildew isolates, whereas the other lines show to be susceptible to the downy mildew isolates.

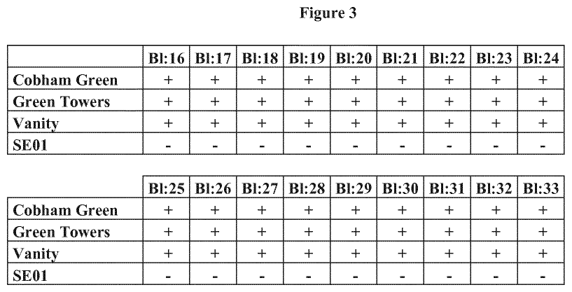

FIG. 4: shows a table of the mutations of present invention and their positions in the MACPF1R protein (SEQ ID NO: 4).

FIG. 5: shows the alignment of the amino acid sequence of MACPF1 (SEQ ID NO: 2) and the MACPF1R (SEQ ID NO: 4) protein. Differences between the two protein sequences have been indicated in grey and correspond with the information as presented in FIG. 4.

FIG. 6: shows the cDNA sequence (SEQ ID NO: 1) encoded by the MACPF1 gene of Lactuca sativa.

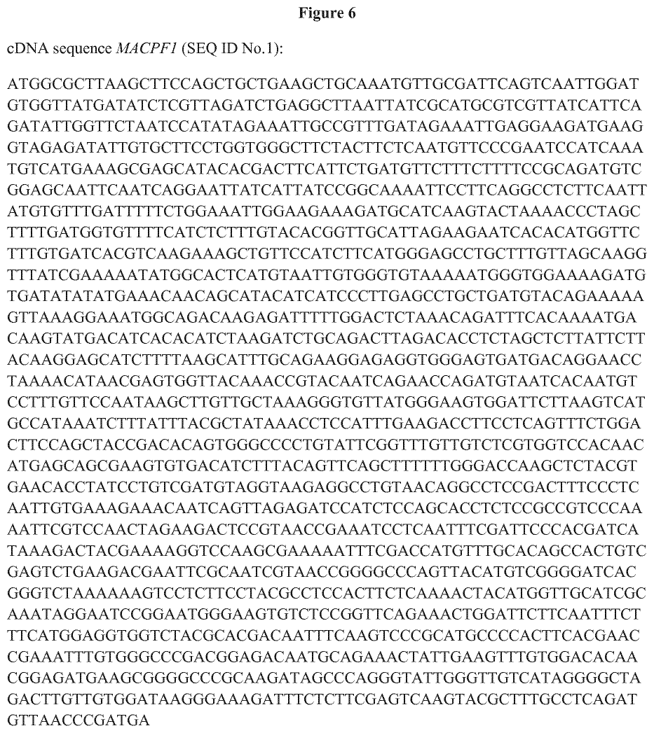

FIG. 7: shows the protein sequence (SEQ ID NO: 2) encoded by the MACPF1 gene of Lactuca sativa.

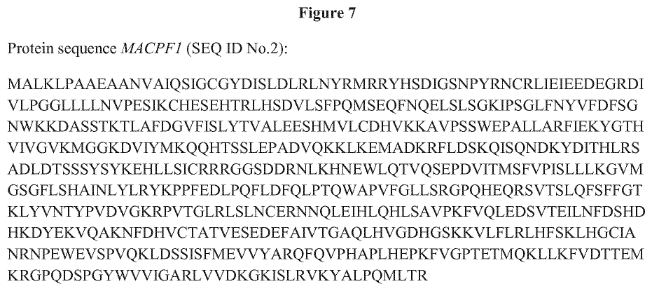

FIG. 8: shows the cDNA sequence (SEQ ID NO: 3) encoded by the MACPF1R gene of Lactuca serriola.

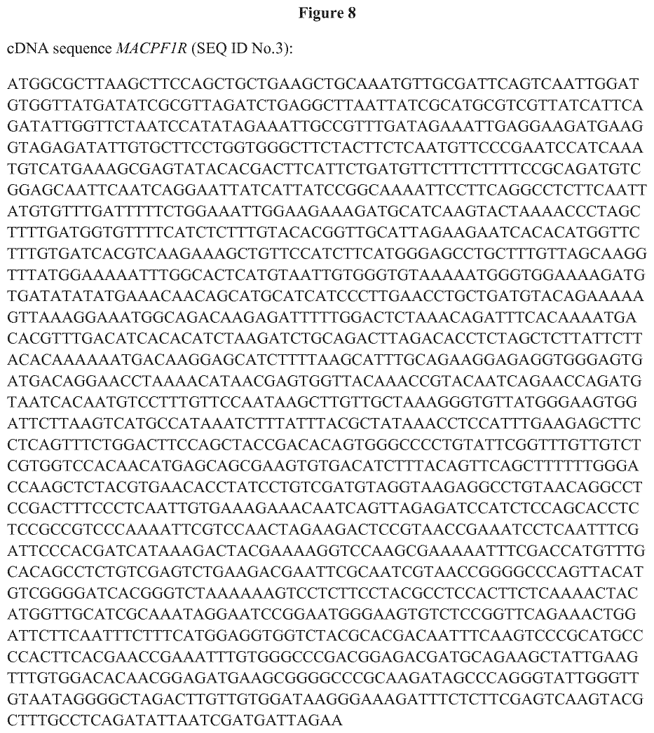

FIG. 9: shows the protein sequence (SEQ ID NO: 4) encoded by the MACPF1R gene of Lactuca serriola.

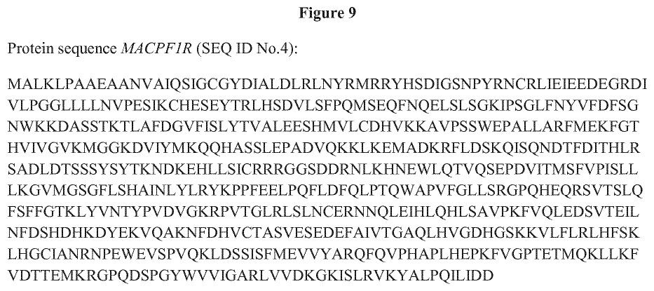

### DETAILED DESCRIPTION

Specifically, the above object, amongst other objects, is met, according to a first aspect, by the present invention by a lettuce plant that is resistant to downy mildew, wherein said plant comprises one or more mutations in a MACPF1 gene, wherein said MACPF1 gene encodes for a protein sequence having at least 90% sequence identity with SEQ ID NO: 2, preferably at least 95%, more preferably at least 98%, most preferably at least 99%.

Research on the MACPF (Membrane Attack Complex/Perforin) superfamily is mainly focused on its function in humans where it plays a role in the immune system in defeating virus and bacteria related diseases. The MACPF superfamily is named after a domain that is common to the membrane attack complex (MAC) proteins of complement and Perforin. Many members are important pore forming toxins in eukaryotes. The archetypal members of the family are complement C9 and Perforin, both of which function in human immunity. C9 functions by punching holes in the membranes of Gram-negative bacteria. Perforin is released by cytotoxic T cells and lyses virally infected and transformed cells. In addition Perforin permits delivery of cytotoxic proteases called granzymes that cause cell death.

The majority of disease resistance genes in plants encode nucleotide-binding site leucine-rich repeat proteins, also known as NBS-LRR proteins (encoded by R genes). These proteins are characterized by nucleotide-binding site (NBS) and leucine-rich repeat (LRR) domains as well as variable amino-and carboxy-terminal domains and are involved in the detection of diverse pathogens, including bacteria, viruses, fungi, nematodes, insects and oomycetes. There are two major subfamilies of plant NBS-LRR proteins defined by the Toll/interleukin-1 receptor (TIR) or the coiled-coil (CC) motifs in the amino-terminal domain and are both involved in pathogen recognition. The MACPF1R gene is involved in a completely different mechanism than the known NBS-LRR mechanism (R genes) in the plant. Therefore, the presence of the MACPF1R resistance gene will decrease the chances of the pathogen overcoming the resistance, as often seen with the R genes. Even so, combined with R genes, disease resistance (e.g. against downy mildew) may even be further improved.

The identification of a novel candidate dominant resistance gene, indicated here as the MACPF1R gene is obtained by gene mapping of multiple independent downy mildew resistance genes in Lettuce. For the first time a MACPF gene has been found in plants that can be linked to plant disease resistance. Using gene mapping methods a gene region was disclosed in lettuce which hosts a number of novel annotated genes that are associated with pathogen resistance, called the membrane attack complex and Perforin (MACPF) gene. In lettuce there are five MACPF homologs present in the lettuce genome, 4 are clustered on chromosome 9 and one is present on chromosome 7. Only one of them is involved with this Bremia resistance phenotype, MACPF1R. This MACPF1R gene of present invention gives resistance to all Bremia races B1 1 to 33, preferably BI 1 to 35, more preferably a broad spectrum Bremia lactucae resistance.

To demonstrate that the MACPF1 gene family is related to Bremia resistance, the putative resistance genes (MACPF1R) have been silenced by tobacco rattle virus (TRV)-based virus-induced gene silencing (VIGS) to induce susceptibility to B. lactucae infection in resistant L. serriola lettuce lines containing the MACPFR1 resistance gene. With VIGS it was demonstrated that the MACPF1R gene was associated with downy mildew resistance, VIGS gene silencing was used to create Bremia-susceptibility in resistant Lactuca species. Resistant lettuce plants were transient transformed with a MACPF1R silencing construct. With VIGS, resistant lettuce lines (L. serriola) were made susceptible by removing the MACPF1R gene via virus induced gene silencing, thereby silencing the MACPF1R gene.

Furthermore it was shown by stable transformation of MACPF1R gene in the susceptible parent (L. sativa) that when MACPF1R was segregating in the next generation it resulted in resistant plants when MACPF1R was present.

According to another preferred embodiment, the present invention relates to the Lettuce plant, wherein the mutations in the MACPF1 gene result in amino acid changes comprised of amino acid substitutions on the amino acid positions 25, 84, 178, 181, 204, 235, 236, 329, 450, 586, 588, and 589 in the MACPF1 protein represented by SEQ ID NO: 2.

According to yet another preferred embodiment, the present invention relates to the Lettuce plant, wherein the mutations in the MACPF1 gene result in amino acid changes further comprised of an addition on amino acid position 590 in the MACPF protein represented by SEQ ID NO: 2.

According to yet another preferred embodiment, the present invention relates to the Lettuce plant, wherein the mutations in the MACPF1 gene result in amino acid changes are further comprised of an insertion of four amino acids on the positions 255 to 258 in the MACPF1 protein represented by SEQ ID NO: 2.

According to another preferred embodiment, the present invention relates to the Lettuce plant, wherein the MACPF1 gene that comprises one or more mutations encodes for the protein sequence represented by SEQ ID NO: 4. Sequencing experiments showed that the protein encoded by the MACPF1R gene from the resistant plant compared with the protein encoded by the MACPF gene of a plant that is susceptible differs in 12 amino acid substitutions, one amino acid addition and an insertion of 4 amino acids; the gene (MACPF1R) that encodes for the protein that comprises all the above mutations is represented by SEQ ID NO: 3. The mutated protein is represented by SEQ ID NO: 4.

According to yet another preferred embodiment, the present invention relates to the lettuce plant, wherein the plant is selected from Lactuca sativa, Lactuca virosa, Lactuca saligna, Lactuca serriola, Lactuca aculeate, Lactuca georgica, Lactuca perennis, Lactuca tatarica, Lactuca viminea, preferably Lactuca sativa.

According to a preferred embodiment, the present invention relates to the lettuce plant, wherein the mutations in the MACPF1 gene are obtained by gene editing techniques, preferably by mutagenesis and/or CRISPR/Cas.

According to another preferred embodiment, the present invention relates to the Lettuce plant, wherein the downy mildew is caused by an oomycete, more preferably Bremia lactucae.

According to another preferred embodiment, the present invention relates to the lettuce plant, wherein the lettuce plant is resistant to downy mildew caused by one or more of Bremia lactucae selected from the group of race B11 to B133. The resistant lettuce plant of present invention is resistance to all Bremia races B11 to B133, preferably B11 to B135, more preferably broad spectrum Bremia lactucae resistant.

According to yet another preferred embodiment, the present invention relates to the lettuce plant, wherein the resistance gene MACPF1R is obtainable from deposit number NCIMB 42435.

The present invention, according to a second aspect, relates to seed produced by the lettuce plant of present invention.

The present invention, according to a third aspect, relates to a resistance gene MACPF1R that confers broad spectrum resistance to oomycetes in lettuce plants, wherein the gene comprises a coding sequence that has at least 90% sequence identity with SEQ ID NO: 3, preferably at least 95%, more preferably at least 98%, most preferably at least 99%, most preferably 100%. SEQ ID NO: 3 represents the coding nucleotide sequence of MACPF1R resistance gene of Lactuca serriola and encodes for the MACPF1R protein sequence represented by SEQ ID NO: 4. SEQ ID NO: 4 represents the MACPF1R protein sequence of Lactuca serriola and lettuce plants that express this protein show complete resistance to downy mildew.

According to a preferred embodiment, the present invention relates to resistance gene MACPF1R, wherein the gene encodes for a MACPF1R protein that has at least 85% sequence identity with SEQ ID NO: 4, preferably at least 90%, more preferably at least 95%, most preferably at least 98%, most preferably 100%.

According to another preferred embodiment, the present invention relates to the resistance gene MACPF1R, wherein broad spectrum resistance to oomycetes in lettuce comprises resistance to Bremia lactucae of race B11 to B133.

According to yet another preferred embodiment, the present invention relates to the resistance gene MACPF1R, wherein the plant is selected from Lactuca sativa, Lactuca virosa, Lactuca saligna, Lactuca serriola, Lactuca aculeate, Lactuca georgica, Lactuca perennis, Lactuca tatarica, Lactuca viminea, preferably Lactuca sativa.

The present invention, according to a further aspect, relates to a seed produced by a lettuce plant of present invention.

The present invention, according to a further aspect, relates to a method for obtaining a lettuce plant that is resistant to downy mildew, wherein the method comprises the steps of,
a) crossing a lettuce plant comprised of the resistance gene MACPF1R of present invention with a lettuce plant that is not resistant to oomycetes,
b) optionally, selfing the plant obtained in step a) for at least one time,
c) selecting the plants that are resistant to downy mildew.
   In the method of present invention the lettuce plant is selected from Lactuca sativa, Lactuca virosa, Lactuca saligna, Lactuca serriola, Lactuca aculeate, Lactuca georgica, Lactuca perennis, Lactuca tatarica, Lactuca viminea, preferably Lactuca sativa.

The present invention, according to a further aspect, relates to a method for obtaining a lettuce plant that is resistant to downy mildew, wherein the method comprises the step of providing one or more mutations in a MACPF1 gene of a lettuce plant, resulting in a MACPF1R resistance gene of present invention. The MACPF1 gene comprises a coding sequence that has at least 90% sequence identity with SEQ ID NO: 1, preferably at least 95%, more preferably at least 98%, most preferably at least 99%, most preferably 100%. SEQ ID NO: 1 represents the coding nucleotide sequence of the MACPF1 gene of Lactuca sativa. This sequence is the wild type sequence and does not contain the mutations as compared to the resistance (MACPF1R) gene of present invention.

According to another preferred embodiment, the present invention relates to the method, wherein the mutations in the MACPF1 gene result in amino acid changes comprised of amino acid substitutions on the amino acid positions 25, 84, 178, 181, 204, 235, 236, 329, 450, 586, 588, and 589 in the MACPF1 protein represented by SEQ ID NO: 2. SEQ ID NO: 2 represents the MACPF1 protein sequence of Lactuca sativa. This protein sequence does not comprise the mutations as compared to the MACPF1R protein of present invention. Therefore, L. sativa that express the protein of SEQ ID NO: 2 is susceptible to downy Mildew. SEQ ID NO: 2 represent the wild type protein sequence as found in lettuce (Lactuca sativa) that does not contain the mutations that result into the MACPF1R protein (SEQ ID NO: 4). Preferably the amino acid substitutions are S->A, H->Y, I->M, Y->F, T->A, K->T, Y->F, D->E, T->S, M->I, T->I, R->D, respectively on the amino acid position 25, 84, 178, 181, 204, 235, 236, 329, 450, 586, 588, and 589 in the MACPF1 protein. Table 8 shows an overview of the mutations in the MACPF1 protein in their respective positions. The mutated MACPF1 protein (MACPF1R) is represented by SEQ ID NO: 4.

According to yet another preferred embodiment, the present invention relates to the method, wherein the mutations in the MACPF1 gene result in amino acid changes further comprised of an addition, preferably of Aspartic Acid (D), on amino acid position 590 in the MACPF1 protein represented by SEQ ID NO: 2.

According to a preferred embodiment, the present invention relates to the method, wherein the mutations in the MACPF1 gene result in amino acid changes further comprised of an insertion of four amino acids on the positions 255 to 258 in the MACPF1 protein represented by SEQ ID NO: 2. The insertion of four amino acids is preferably TKND (SEQ ID NO: 13).

According to a preferred embodiment, the present invention relates to the method, wherein the mutations in the MACPF1 gene results in a protein represented by SEQ ID NO: 4.

According to another preferred embodiment, the present invention relates to the method, wherein the lettuce plant is selected from Lactuca sativa, Lactuca virosa, Lactuca saligna, Lactuca serriola, Lactuca aculeate, Lactuca georgica, Lactuca perennis, Lactuca tatarica, Lactuca viminea, preferably Lactuca sativa.

A lettuce plant comprised of the insertion of 4 amino acids in combination with an addition, in combination with the amino acid substitutions gives a high downy mildew resistance phenotype. A plant having this resistant phenotype can be obtained via use of gene editing and/or mutation techniques, such as EMS mutagenesis or CRISPR/Cas in concert with cloning techniques on the MACPF1 gene to generate disease resistant crops.

According to yet another preferred embodiment, the present invention relates to the method, wherein the mutations in the MACPF1 gene are obtained by gene editing techniques, preferably by mutagenesis and/or CRISPR/Cas. Alternatively, a MACPF1R gene can be brought into the plant by means of transgenic techniques or by introgression.

According to another preferred embodiment, the present invention relates to the method, wherein the mutations in the MACPF1 gene are non-natural mutations. Mutations induced by gene editing techniques such as mutagenesis, CRISPR/Cas, transgenic techniques, or others can be regarded as non-natural mutations.

The present invention, according to a further aspect, relates to the use of a plasmid for introducing a resistance gene into the genome of a plant or plant cell, wherein the plasmid comprises the resistance gene MACPF1R of present invention. The resistance gene of present invention may be transferred (e.g. by transformation or transfection) into plants, such as lettuce plants, using a plasmid that comprises the the MACPF1R resistance gene of present invention wherein the gene comprises a coding sequence that has at least 90% sequence identity with SEQ ID NO: 3. The resistance gene MACPF1R encodes for a MACPF1R protein that has at least 85% sequence identity with SEQ ID NO: 4. The Resistance gene MACPF1R , after being transferred into the plant would provide broad spectrum resistance to oomycetes, i.e. resistance to Bremia lactucae of race B11 to B133.

### EXAMPLES

### Synthesis of Construct MACPF1R

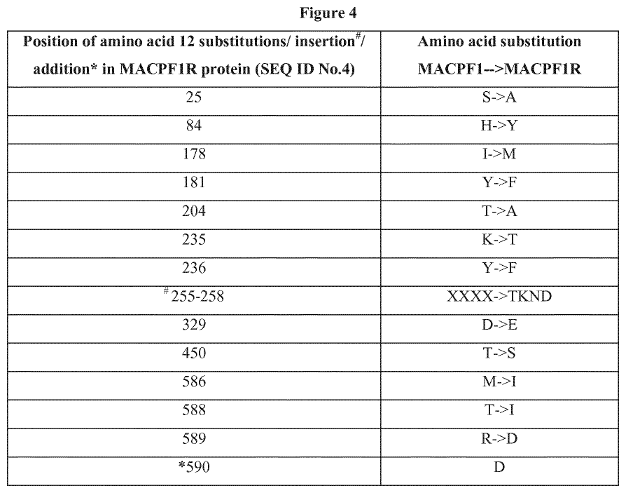

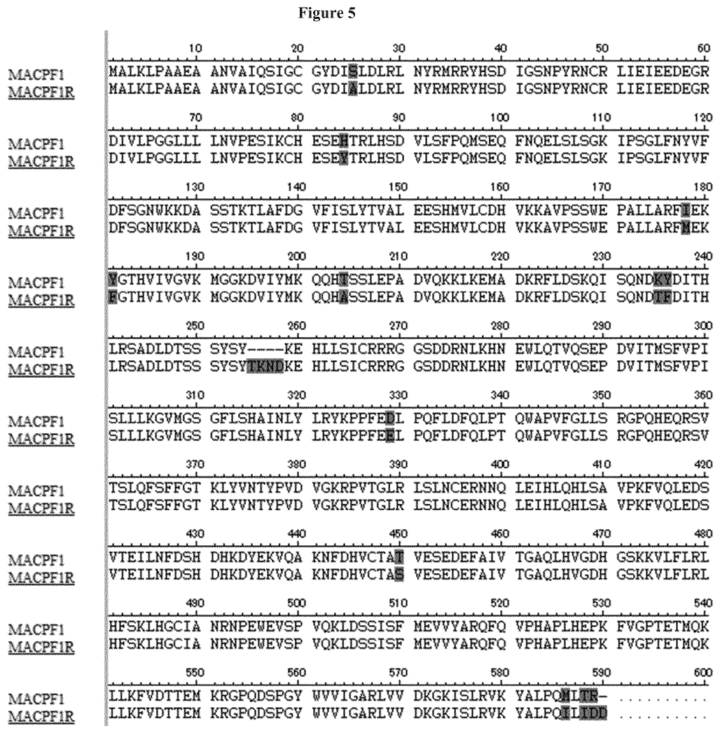

In order to study the function of the MACPF1R gene and more specifically if the amino acid substitutions, additions and/or the amino acid insertion are causing the resistance, the MACPF1R construct has been developed. To study if the insertion or the amino acid substitution +addition is effecting resistance, three constructs were made: one with the resistance gene of present invention, one construct with the insertion and no substitutions or additions called LsMACPF1Ins and one construct with the substitution +addition and not the insertion called LsMACPF1Sub. Synthetically constructs with gateway sites were made by Gen9. These fragments were cloned into the vector pK7WG2,0 and transformed to A.tum GV2260. Finally those constructs were stably transformed into L. sativa cultivars Cobham Green and Wendel. The differences between the MACPF1 and MACPF1R protein are 12 amino acid substitutions, an amino acid addition and an insertion of 4 amino acids, see FIG. 4 and FIG. 5 for the specific mutations in the protein and their positions.

### Transformation into Lettuce to Study MACPF1R Function

The multiple constructs of above of the MACPF1 gene were transformed into lettuce (L. sativa) using co-cultivation with agrobacterium. The following construct were used:
1) The MACPF1R gene, called “LsMACPF1R” (=MACPF1 gene wherein insertion, addition and substitutions are present),
2) The MACPF1R gene without the insertion, called “LsMACPF1Sub”,
3) The MACPF1R gene without substitutions and addition, called “LsMACPF1Ins”.

Furthermore, it was shown by stable transformation of MACPF1R gene in the susceptible parent (L. sativa) that when MACPF1R was segregating in the next generation it resulted in resistant plants when the MACPF1R resistance gene was present. This was followed by primers specific for the MACPF1R gene. Sequences are present in table 1. Plants were selected based on the primers below in table 1 (SEQ ID NO: 5 and SEQ ID NO: 6, respectively).

TABLE 1

| Primer name | Sequence |
|---|---|
| MACPF1R_F | 5′- TTTCACAAAATGACACGTTTGAC -3′ |
| | (SEQ ID NO: 5) |
| MACPF1R_R | 5′- TGCTTAAAAGATGCTCCTTGTC -3′ (SEQ |
| | ID NO: 6) |

### MACPF1R Silencing Experiment Using Virus Induced Gene Silencing (VIGS)

Tobacco rattle virus (TRV)-derived VIGS vectors have been abundantly described to study gene function in Arabidopsis thaliana, Nicotiana benthamiana, Lycopersicon esculentum and other plants (see for example Huang C, Qian Y, Li Z, Zhou X .: Virus-induced gene silencing and its application in plant functional genomics. Sci China Life Sci. 2012;55(2):99-108). Briefly, lines containing MACPF1R were silenced for MACPF1 by VIGS. Independent of MACPF1R silencing the PDS gene is silenced as well that serves as positive control to indicate if VIGS is working. PDS is involved in carotenoid biosynthesis and is the first step in lycopene biosynthesis. This step is catalyzed by phytoene desaturase (PDS). When silencing of the PDS gene is achieved, this results in bleached leaves. Silencing of MACPF1R did not result in a visual phenotype. Therefore, all plants that were MACPF1-VIGS inoculated were harvested and put in a tray and sprayed with Bremia B130. This resulted in susceptible leaves while non-silenced MACPF1 plants stayed resistant.

### Resistance Test/Biotest for Downy Mildew in Lettuce

The MACPF1 constructs (LsMACPF1R,-Ins and-Sub) were introduced in lettuce lines using co-cultivation with agrobacterium to get stable transformants. Introducing of the MACPF1R consensus sequence stable transformed in Bremia susceptible lettuce lines (Cobham Green and Wendel) result in Bremia resistant lines in T0, T1 and T2 generation. Outcome of the results are lettuce T1 plants containing the different constructs which are tested for resistance to the oomycete Bremia.

For LsMACPF1 Ins, 47 independent lines in the Cobham Green background and 9 independent lines in the Wendel background were made. In the case of LsMACPF1Sub 57 independent lines in Cobham Green background and 8 independent lines in the Wendel background were made. The seeds of those independent lines were tested in a Bremia seedling test in which 50 seeds per transformant were inoculated with Bremia. The results are that all plants (Wendel and Cobham Green) were susceptible for Bremia (B124 Bremia tested in Wendel, B124 and BL32 Bremia tested in Cobham Green).

The above experiments indicate that both substitutions, addition and the insertion in the MACPF1 gene/protein are necessary to provide the full resistant phenotype to Bremia. If we isolate only the insertion or only the substitution+addition from the resistant source, the Bremia resistance is lost. Therefore the substitutions, addition and insertion are needed to be present in the MACPF1 protein to make an active MACPF1R protein to form pores which could give resistance to Bremia in lettuce.

Leaves of resistant plants stably transformed with or without VIGS MACPF1, were put in trays with moistened paperboard. The infected seedlings are suspended in 20 mL water, filtered by cheesecloth and the flow-through is collected in a spray flask. One tray is spray-inoculated with this B. lactucae suspension. The trays are covered with a glass plate and stored in a climate chamber at 15° C. (12 hours of light). A black, opaque foil is placed over the trays for one day to improve growth of B. lactucae. After one day, the foil is removed. Eight to ten days after infection leaves are phenotypically scored by eye on the presence of Bremia and qPCR was performed to determine MACPF1R expression.

### Expression of MACPF1R Genes in Lettuce

A number of gene expression experiments were conducted in lettuce tissues obtained form the VIGS experiment as outlined above, to determine MACPF1R expression. The response of lettuce leaf discs to Bremia lactucae infection was examined and gene expression studies were used to assess VIGS analysis.

To create more insight in the response of lettuce to infection with Bremia (Bremia lactucae), leaves of resistant and susceptible plants were harvested. cDNA was synthesized from RNA that had been isolated from infected leaf discs. The expression of MACPF1 was assessed in lettuce by conducting qPCR. Expression of Bremia lactucae actin and expression of MACPF1 were analyzed by qPCR using the primers as set out in Table 2 (SEQ ID NO: 6, SEQ ID NO: 7, SEQ ID NO: 8, SEQ ID NO: 9, SEQ ID NO: 10, SEQ ID NO: 11, and SEQ ID NO: 12, respectively). This experiment was performed independent in duplo.

TABLE 2

| Primer name | Sequence |
|---|---|
| MACPF 1 Fwd | 5′-ACATCACACATCTAAGATCTGC-3′ (SEQ |
| | ID NO: 7) |
| MACPF 1 Rv | 5′-ATGGAGGTTTATAGCGTAAATA-3′ (SEQ |
| | ID NO: 8) |
| B. lactucae | 5′-GCGAGAAATTGTGCGTGATA-3′ (SEQ ID |
| Actin Fwd | NO: 9) |
| B. lactucae | 5′-ACTCGGCTGCAGTCTTCATT-3′ (SEQ ID |
| Actin Rv | NO: 10) |
| LsTUA-3F | 5′-CTTCTTAGTGTTCAATGCTGTTGG-3′ |
| | (SEQ ID NO: 11) |
| LsTUA-3R | 5′-GAAGGGTAGATAGTGAAACCGAGC-3′ |
| | (SEQ ID NO: 12) |

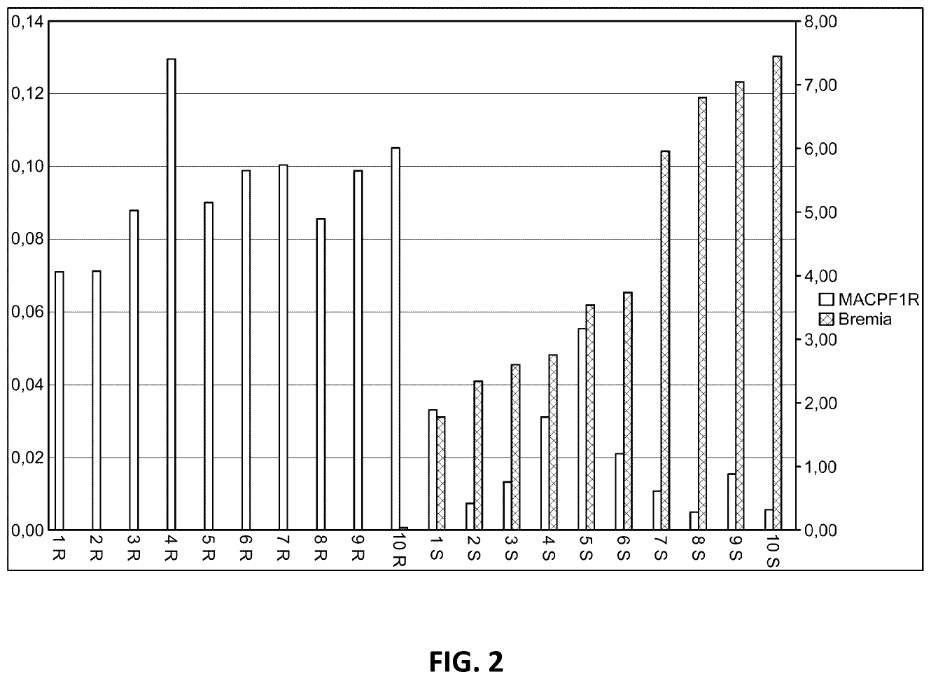

Results (FIG. 2) shows that in leaves of the plants that are resistant to Bremia little to no Bremia was detected and that the level of MACPF1R was very high. The leaves that originate from plants that are susceptible to Bremia, showed the opposite pattern, a high level of Bremia and low levels of MACPF1R expression.

1-25. (canceled)

26. A lettuce plant comprising a MACPF1R gene encoding a protein sequence having at least 90% sequence identity to SEQ ID NO: 4 and comprising alanine at position 25, tyrosine at position 84, methionine at position 178, phenylalanine at position 181, alanine at position 204, threonine at position 235, phenylalanine at position 236, threonine, lysine, asparagine, aspartic acid at positions 255-258, glutamic acid at position 329, serine at position 450, isoleucine at position 586, isoleucine at position 588, and aspartic acid at position 589, wherein the lettuce plant is not Lactuca serriola.

27. The lettuce plant of claim 26, wherein the lettuce plant is selected from Lactuca sativa, Lactuca virosa, Lactuca saligna, Lactuca aculeate, Lactuca georgica, Lactuca perennis, Lactuca tatarica, and Lactuca viminea.

28. The lettuce plant of claim 26, wherein the lettuce plant is resistant to downy mildew.

29. The lettuce plant of claim 28, wherein the downy mildew is caused by Bremia lactucae.

30. The lettuce of claim 28, wherein the downy mildew is caused by one or more of Bremia lactucae races B11 to B133.

31. The lettuce plant of claim 28, wherein the protein sequence further comprises aspartic acid at position 590.

32. The lettuce plant of claim 26, wherein the MACPF1R gene encodes a protein sequence having at least 95% sequence identity to SEQ ID NO: 4.

33. The lettuce plant of claim 26, wherein the MACPF1R gene encodes a protein sequence having at least 98% sequence identity to SEQ ID NO: 4.

34. A lettuce seed comprising a MACPF1R gene encoding a protein sequence having at least 90% sequence identity to SEQ ID NO: 4 and comprising alanine at position 25, tyrosine at position 84, methionine at position 178, phenylalanine at position 181, alanine at position 204, threonine at position 235, phenylalanine at position 236, threonine, lysine, asparagine, aspartic acid at positions 255-258, glutamic acid at position 329, serine at position 450, isoleucine at position 586, isoleucine at position 588, and aspartic acid at position 589, wherein the lettuce seed is not Lactuca serriola.

35. The lettuce seed of claim 34, wherein the lettuce seed is selected from Lactuca sativa, Lactuca virosa, Lactuca saligna, Lactuca aculeate, Lactuca georgica, Lactuca perennis, Lactuca tatarica, and Lactuca viminea.

36. The lettuce seed of claim 34, wherein the protein sequence further comprises aspartic acid at position 590.

37. The lettuce seed of claim 34, wherein the MACPF1R gene encodes a protein sequence having at least 95% sequence identity to SEQ ID NO: 4.

38. The lettuce seed of claim 34, wherein the MACPF1R gene encodes a protein sequence having at least 98% sequence identity to SEQ ID NO: 4.

39. A method for obtaining a lettuce plant that is resistant to downy mildew, wherein the method comprises the steps of,
a) crossing a lettuce plant comprising a MACPF1R gene encoding a protein sequence having at least 90% sequence identity to SEQ ID NO: 4 and comprising alanine at position 25, tyrosine at position 84, methionine at position 178, phenylalanine at position 181, alanine at position 204, threonine at position 235, phenylalanine at position 236, threonine, lysine, asparagine, aspartic acid at positions 255-258, glutamic acid at position 329, serine at position 450, isoleucine at position 586, isoleucine at position 588, and aspartic acid at position 589 with a lettuce plant that is not resistant to oomycetes,
b) optionally, selfing the plant obtained in step a) for at least one time, and
c) selecting offspring plants that are resistant to downy mildew.

40. The method of claim 39, wherein the protein sequence comprises aspartic acid at position 590.

41. The method of claim 39, wherein the MACPF1R gene encodes a protein sequence having at least 95% sequence identity to SEQ ID NO: 4.

42. The method of claim 39, wherein the MACPF1R gene encodes a protein sequence having at least 98% sequence identity to SEQ ID NO: 4.

43. A method for obtaining a lettuce plant that is resistant to downy mildew, wherein the method comprises a step of providing one or more mutations in a MACPF1 gene of a lettuce plant, resulting in a MACPF1R gene that encodes for a protein sequence having at least 90% sequence identity to SEQ ID NO: 4 and comprising alanine at position 25, tyrosine at position 84, methionine at position 178, phenylalanine at position 181, alanine at position 204, threonine at position 235, phenylalanine at position 236, threonine, lysine, asparagine, aspartic acid at positions 255-258, glutamic acid at position 329, serine at position 450, isoleucine at position 586, isoleucine at position 588, and aspartic acid at position 589.

44. The method of claim 43, wherein the lettuce plant is selected from Lactuca sativa, Lactuca virosa, Lactuca saligna, Lactuca aculeate, Lactuca georgica, Lactuca perennis, Lactuca tatarica, and Lactuca viminea.

45. The method of claim 43, wherein the mutations in the MACPF1 gene are obtained by gene editing techniques.

46. The method of claim 43, wherein the protein sequence further comprises aspartic acid at position 590.

47. The method of claim 43, wherein the MACPF1R gene encodes a protein sequence having at least 95% sequence identity to SEQ ID NO: 4.

48. The method of claim 43, wherein the MACPF1R gene encodes a protein sequence having at least 98% sequence identity to SEQ ID NO: 4.


SEQUENCE LISTING (13 sequences)

SEQ ID NO: 1 - DNA, 1758 bp, Lactuca sativa
  source 1..1758: mol_type = unassigned DNA
atggcgcttaagcttccagctgctgaagctgcaaatgttgcgattcagtcaattggatgtggttatgatatctcgttagatctgaggcttaattatcgcatgcgtcgttatcattcagatattggttctaatccatatagaaattgccgtttgatagaaattgaggaagatgaaggtagagatattgtgcttcctggtgggcttctacttctcaatgttcccgaatccatcaaatgtcatgaaagcgagcatacacgacttcattctgatgttctttcttttccgcagatgtcggagcaattcaatcaggaattatcattatccggcaaaattccttcaggcctcttcaattatgtgtttgatttttctggaaattggaagaaagatgcatcaagtactaaaaccctagcttttgatggtgttttcatctctttgtacacggttgcattagaagaatcacacatggttctttgtgatcacgtcaagaaagctgttccatcttcatgggagcctgctttgttagcaaggtttatcgaaaaatatggcactcatgtaattgtgggtgtaaaaatgggtggaaaagatgtgatatatatgaaacaacagcatacatcatcccttgagcctgctgatgtacagaaaaagttaaaggaaatggcagacaagagatttttggactctaaacagatttcacaaaatgacaagtatgacatcacacatctaagatctgcagacttagacacctctagctcttattcttacaaggagcatcttttaagcatttgcagaaggagaggtgggagtgatgacaggaacctaaaacataacgagtggttacaaaccgtacaatcagaaccagatgtaatcacaatgtcctttgttccaataagcttgttgctaaagggtgttatgggaagtggattcttaagtcatgccataaatctttatttacgctataaacctccatttgaagaccttcctcagtttctggacttccagctaccgacacagtgggcccctgtattcggtttgttgtctcgtggtccacaacatgagcagcgaagtgtgacatctttacagttcagcttttttgggaccaagctctacgtgaacacctatcctgtcgatgtaggtaagaggcctgtaacaggcctccgactttccctcaattgtgaaagaaacaatcagttagagatccatctccagcacctctccgccgtcccaaaattcgtccaactagaagactccgtaaccgaaatcctcaatttcgattcccacgatcataaagactacgaaaaggtccaagcgaaaaatttcgaccatgtttgcacagccactgtcgagtctgaagacgaattcgcaatcgtaaccggggcccagttacatgtcggggatcacgggtctaaaaaagtcctcttcctacgcctccacttctcaaaactacatggttgcatcgcaaataggaatccggaatgggaagtgtctccggttcagaaactggattcttcaatttctttcatggaggtggtctacgcacgacaatttcaagtcccgcatgccccacttcacgaaccgaaatttgtgggcccgacggagacaatgcagaaactattgaagtttgtggacacaacggagatgaagcggggcccgcaagatagcccagggtattgggttgtcataggggctagacttgttgtggataagggaaagatttctcttcgagtcaagtacgctttgcctcagatgttaacccgatga

SEQ ID NO: 2 - protein, 585 aa, Lactuca sativa
MALKLPAAEAANVAIQSIGCGYDISLDLRLNYRMRRYHSDIGSNPYRNCRLIEIEEDEGRDIVLPGGLLLLNVPESIKCHESEHTRLHSDVLSFPQMSEQFNQELSLSGKIPSGLFNYVFDFSGNWKKDASSTKTLAFDGVFISLYTVALEESHMVLCDHVKKAVPSSWEPALLARFIEKYGTHVIVGVKMGGKDVIYMKQQHTSSLEPADVQKKLKEMADKRFLDSKQISQNDKYDITHLRSADLDTSSSYSYKEHLLSICRRRGGSDDRNLKHNEWLQTVQSEPDVITMSFVPISLLLKGVMGSGFLSHAINLYLRYKPPFEDLPQFLDFQLPTQWAPVFGLLSRGPQHEQRSVTSLQFSFFGTKLYVNTYPVDVGKRPVTGLRLSLNCERNNQLEIHLQHLSAVPKFVQLEDSVTEILNFDSHDHKDYEKVQAKNFDHVCTATVESEDEFAIVTGAQLHVGDHGSKKVLFLRLHFSKLHGCIANRNPEWEVSPVQKLDSSISFMEVVYARQFQVPHAPLHEPKFVGPTETMQKLLKFVDTTEMKRGPQDSPGYWVVIGARLVVDKGKISLRVKYALPQMLTR

SEQ ID NO: 3 - DNA, 1775 bp, Lactuca serriola
  source 1..1775: mol_type = unassigned DNA
atggcgcttaagcttccagctgctgaagctgcaaatgttgcgattcagtcaattggatgtggttatgatatcgcgttagatctgaggcttaattatcgcatgcgtcgttatcattcagatattggttctaatccatatagaaattgccgtttgatagaaattgaggaagatgaaggtagagatattgtgcttcctggtgggcttctacttctcaatgttcccgaatccatcaaatgtcatgaaagcgagtatacacgacttcattctgatgttctttcttttccgcagatgtcggagcaattcaatcaggaattatcattatccggcaaaattccttcaggcctcttcaattatgtgtttgatttttctggaaattggaagaaagatgcatcaagtactaaaaccctagcttttgatggtgttttcatctctttgtacacggttgcattagaagaatcacacatggttctttgtgatcacgtcaagaaagctgttccatcttcatgggagcctgctttgttagcaaggtttatggaaaaatttggcactcatgtaattgtgggtgtaaaaatgggtggaaaagatgtgatatatatgaaacaacagcatgcatcatcccttgaacctgctgatgtacagaaaaagttaaaggaaatggcagacaagagatttttggactctaaacagatttcacaaaatgacacgtttgacatcacacatctaagatctgcagacttagacacctctagctcttattcttacacaaaaaatgacaaggagcatcttttaagcatttgcagaaggagaggtgggagtgatgacaggaacctaaaacataacgagtggttacaaaccgtacaatcagaaccagatgtaatcacaatgtcctttgttccaataagcttgttgctaaagggtgttatgggaagtggattcttaagtcatgccataaatctttatttacgctataaacctccatttgaagagcttcctcagtttctggacttccagctaccgacacagtgggcccctgtattcggtttgttgtctcgtggtccacaacatgagcagcgaagtgtgacatctttacagttcagcttttttgggaccaagctctacgtgaacacctatcctgtcgatgtaggtaagaggcctgtaacaggcctccgactttccctcaattgtgaaagaaacaatcagttagagatccatctccagcacctctccgccgtcccaaaattcgtccaactagaagactccgtaaccgaaatcctcaatttcgattcccacgatcataaagactacgaaaaggtccaagcgaaaaatttcgaccatgtttgcacagcctctgtcgagtctgaagacgaattcgcaatcgtaaccggggcccagttacatgtcggggatcacgggtctaaaaaagtcctcttcctacgcctccacttctcaaaactacatggttgcatcgcaaataggaatccggaatgggaagtgtctccggttcagaaactggattcttcaatttctttcatggaggtggtctacgcacgacaatttcaagtcccgcatgccccacttcacgaaccgaaatttgtgggcccgacggagacgatgcagaagctattgaagtttgtggacacaacggagatgaagcggggcccgcaagatagcccagggtattgggttgtaataggggctagacttgttgtggataagggaaagatttctcttcgagtcaagtacgctttgcctcagatattaatcgatgattagaa

SEQ ID NO: 4 - protein, 590 aa, Lactuca serriola
MALKLPAAEAANVAIQSIGCGYDIALDLRLNYRMRRYHSDIGSNPYRNCRLIEIEEDEGRDIVLPGGLLLLNVPESIKCHESEYTRLHSDVLSFPQMSEQFNQELSLSGKIPSGLFNYVFDFSGNWKKDASSTKTLAFDGVFISLYTVALEESHMVLCDHVKKAVPSSWEPALLARFMEKFGTHVIVGVKMGGKDVIYMKQQHASSLEPADVQKKLKEMADKRFLDSKQISQNDTFDITHLRSADLDTSSSYSYTKNDKEHLLSICRRRGGSDDRNLKHNEWLQTVQSEPDVITMSFVPISLLLKGVMGSGFLSHAINLYLRYKPPFEELPQFLDFQLPTQWAPVFGLLSRGPQHEQRSVTSLQFSFFGTKLYVNTYPVDVGKRPVTGLRLSLNCERNNQLEIHLQHLSAVPKFVQLEDSVTEILNFDSHDHKDYEKVQAKNFDHVCTASVESEDEFAIVTGAQLHVGDHGSKKVLFLRLHFSKLHGCIANRNPEWEVSPVQKLDSSISFMEVVYARQFQVPHAPLHEPKFVGPTETMQKLLKFVDTTEMKRGPQDSPGYWVVIGARLVVDKGKISLRVKYALPQILIDD

SEQ ID NO: 5 - DNA, 23 bp, synthetic construct
  source 1..23: mol_type = other DNA; note = MACPF1R_F primer
tttcacaaaatgacacgtttgac

SEQ ID NO: 6 - DNA, 22 bp, synthetic construct
  source 1..22: mol_type = other DNA; note = MACPF1R_R primer
tgcttaaaagatgctccttgtc

SEQ ID NO: 7 - DNA, 22 bp, synthetic construct
  source 1..22: mol_type = other DNA; note = MACPF 1 forward primer
acatcacacatctaagatctgc

SEQ ID NO: 8 - DNA, 22 bp, synthetic construct
  source 1..22: mol_type = other DNA; note = MACPF 1 Reverse primer
atggaggtttatagcgtaaata

SEQ ID NO: 9 - DNA, 20 bp, synthetic construct
  source 1..20: mol_type = other DNA; note = B. lactucae actin Forward primer
gcgagaaattgtgcgtgata

SEQ ID NO: 10 - DNA, 20 bp, synthetic construct
  source 1..20: mol_type = other DNA; note = B. lactucae actin Reverse primer
actcggctgcagtcttcatt

SEQ ID NO: 11 - DNA, 24 bp, synthetic construct
  source 1..24: mol_type = other DNA; note = LsTUA-3F primer
cttcttagtgttcaatgctgttgg

SEQ ID NO: 12 - DNA, 24 bp, synthetic construct
  source 1..24: mol_type = other DNA; note = LsTUA-3R primer
gaagggtagatagtgaaaccgagc

SEQ ID NO: 13 - protein, 4 aa, synthetic construct
TKND